(19) United States
(12) Patent Application Publication
(10) Pub. No.: US 2017/0119278 A1
(43) Pub. Date: May 4, 2017

(54) LUMEN TRAVELING DEVICE

(71) Applicant: Elwha LLC, Bellevue, WA (US)

(72) Inventors: Roderick A. Hyde, Redmond, WA (US); Wayne R. Kindsvogel, Seattle, WA (US); Stephen L. Malaska, Redmond, WA (US); Robert C. Petroski, Seattle, WA (US); Katherine E. Sharadin, Redmond, WA (US); Elizabeth A. Sweeney, Seattle, WA (US)

(21) Appl. No.: 14/926,233

(22) Filed: Oct. 29, 2015

(51) Int. Cl. A61B 5/07; A61B 1/00; A61B 5/06; A61B 5/145; A61B 1/04; A61B 5/00

(52) U.S. Cl. CPC A61B 5/07; A61B 1/041; A61B 5/742; A61B 5/065; A61B 5/14539; A61B 5/145; A61B 5/073; A61B 1/00156; A61B 2560/0219; A61B 2562/0219; A61B 2562/162

(57) ABSTRACT

Various embodiments described herein relate to a lumen traveling device and/or system for real-time display of location of the device as it travels through a lumen in a subject's body. In an embodiment, alignment of the externally alignable display and control device with the lumen traveling device located in a lumen (natural or artificial) in a subject's body provides for tracking, memory display, and manipulation of the lumen traveling device.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### BRIEF DESCRIPTION OF THE FIGURES

FIG. 1A is a partial view of a device and system described herein.

FIG. 1B is a partial view of a device and system described herein.

FIG. 1C is a partial view of a device and system described herein.

FIG. 2A is a partial view of a device and system described herein.

FIG. 2B is a partial view of a device and system described herein.

FIG. 3A is a partial view of a device and system described herein.

FIG. 3B is a partial view of a device and system described herein.

FIG. 4 is a partial view of a component of the device and system described herein.

FIG. 5 is a partial view of a component of the device and system described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

In an embodiment, a lumen traveling device and system are adapted for traveling within a natural or artificial (e.g., catheter, shunt) lumen of a subject's body. In an embodiment, the lumen traveling device is at least one of disposable, biodegradable, or bioresorbable, in part or in total. In an embodiment, the lumen traveling device is sized and shaped according to specifications of the particular lumen it is designed to travel within, or the specifications of the function or operation of the lumen traveling device. In an embodiment, the lumen traveling device is of a fixed size or shape. In an embodiment, the lumen traveling device is flexible. In an embodiment, the lumen traveling device is spheroid, cylindrical, pyramidal, cuboid, or any combination thereof. In an embodiment, the lumen traveling device is sized and shaped for ingestion. In an embodiment, the lumen traveling device is sized and shaped for surgical introduction or implantation into a lumen. In an embodiment, the lumen traveling device is sized and shaped for injection into a lumen or an organ having lumen or duct. In an embodiment, the lumen traveling device is sized and shaped for introduction into a lumen via a catheter or cannula. In an embodiment, the lumen traveling device is sized and shaped for injection into at least a portion of an alimentary canal, a blood vessel, a respiratory tract, a urinary tract, genital tract, a duct of an organ, or the like. In an embodiment, the lumen traveling device comprises all or part of a micro-robot (e.g., microbot). In an embodiment, the lumen traveling device includes all or part of a capsule endoscope.

For example, in an embodiment, the lumen traveling device is approximately 50 mm or less, approximately 40 mm or less, approximately 30 mm or less, approximately 20 mm or less, approximately 10 mm or less, approximately 5 mm or less in length. In an embodiment, the lumen traveling device is approximately 20 mm or less, approximately 10 mm or less, approximately 5 mm or less in width or height (i.e. diameter).

In an embodiment, the lumen traveling device includes layers of several materials. In an embodiment, at least one layer of the lumen traveling device includes a permeable or semi-permeable membrane. In an embodiment, the lumen traveling device includes at least one mesh surface. In an embodiment, the lumen traveling device includes expandable or contractable materials, for example metal or plastic components that are capable of being altered in size or shape. In an embodiment, the lumen traveling device includes a shape memory alloy or electroactive polymer. In an embodiment, at least one component of the lumen traveling device includes a metal, ceramic, paper, polymer (plastic, silicone, etc.), silk, or other suitable biocompatible materials. In an embodiment, the lumen traveling device can be manufactured according to various techniques, including 3-D printing, self-assembly, rapid-prototyping, die-cutting, extrusion, injection molding, or the like.

In an embodiment, the lumen traveling device and system includes at least one sensor. In an embodiment, the sensor is used to determine location of the lumen traveling device (e.g., based on the parameters sensed at a particular location of a lumen). As described herein, there is also at least one sensor in the system utilized to determine the location of the EADCD in relation to the lumen traveling device. In an embodiment, at least one sensor is used to determine at least one physiological parameter that may be used for determining a medical treatment, a change in treatment, or a diagnosis of the subject.

In an embodiment, the sensor includes, for example, at least one of a pressure sensor, temperature sensor, flow sensor, viscosity sensor, shear sensor, pH sensor, gas sensor, chemical sensor, optical sensor, acoustic sensor, biosensor, electrical sensor, magnetic sensor, clock, or timer. In an embodiment, the sensor detects a physiological condition, such as level of a blood component (e.g., pH, hormone, vitamin or mineral, cholesterol, oxygen, bilirubin, hemoglobin, etc.), presence or number of a cell type (red blood cells, white blood cells, immune cells, malignant cells, necrotic cells, etc.), immune function (e.g., inflammation, bleeding, infection, auto-immunity, etc.), microbiome (e.g., levels of healthy or unhealthy microorganisms, etc.), blood pressure, or other condition. In an embodiment, the sensor detects lumen surface integrity (e.g., presence of a lesion, tumor, ulcer, fissure, wound, etc.), for example associated with an autoimmune disorder (e.g., Crohn's disease lesion), cancer or precancerous condition (e.g., a tumor or polyp), or vascular disorder (e.g., gastrointestinal bleeding or varices). In an embodiment, the sensor detects an analyte, for example, a physiological analyte. In an embodiment, the sensor detects a tag (e.g., a radiographic or colorimetric agent that binds to cells or components of cells or binds to other components of biological fluids, such as hemoglobin, insulin, etc. and that can be utilized to detect or monitor a specific medical condition or disease).

In an embodiment, the lumen traveling device and/or system includes at least one power source. For example, the power source may be located on the surface of the lumen traveling device, inside a compartment of the lumen traveling device, or other location. In an embodiment, the lumen traveling device and/or system includes at least one battery, microbattery, thin-film battery, or nuclear battery. In an embodiment, the lumen traveling device and/or system includes at least one fuel cell or biofuel cell, for example at least one enzymatic, microbial, or photosynthetic fuel cell. In an embodiment, the power source includes a nanogenerator (e.g. DNA, piezoelectric wires, or other tensile material).

In an embodiment, the power source includes at least one of an optical power source, acoustic receiver, electromagnetic receiver, or electrical power source. In an embodiment, the power source is connected to the lumen traveling device and/or system through a cable or physical link. In an embodiment, the lumen traveling device and/or system is wireless.

In an embodiment, the lumen traveling device (LTD) is located within the subject's body by way of various sensors, as described herein, for example by query sensing (reflected and time-of-flight) or passive sensing (LTD emitting signal) between the LTD and externally alignable display and control device (EADCD). In an embodiment, a specific electromagnetic signal (e.g., RF or magnetic) is coupled between the LTD and EADCD, and the LTD is aligned with the strongest signal, indicating alignment with the EADCD. In an embodiment, the time-of-flight value is utilized to determine the location of the LTD from the EADCD. In an embodiment, the EADCD has more than one receiver at different locations on the device, and a comparison of the strength of the signal at each receiver indicates which receiver the LTD is closer to, and allows for locational determination.

In an embodiment, the lumen traveling device and/or system includes at least one component for harvesting energy. In an embodiment, the lumen traveling device and/or system includes at least one component for harvesting energy from the body, for example kinetic energy (e.g., from fluid flow or peristalsis) or thermal energy, and transducing the energy to power. In an embodiment, the lumen traveling device and/or system includes at least one component for harvesting energy from a source external to the body, for example infrared radiation from a dedicated source.

In an embodiment, the lumen traveling device or system includes at least one component for wireless energy transfer. In an embodiment, the lumen traveling device includes at least one energy receiver configured to receive power from at least one external energy transmitter. For example, acoustic energy, electrical energy, or optical energy can be transmitted to the lumen traveling device from another location. In an embodiment, ultrasonic energy or microwave energy can be beamed to a receiver and converted into a current. In an embodiment, the lumen traveling device includes at least one capacitive coupling link. In an embodiment, the lumen traveling device includes at least one inductive coupling link. In an embodiment, the lumen traveling device can include at least one receiving coil configured to receive energy from an external transmitting coil. In an embodiment, the lumen traveling device includes multiple receiving coils, for example in a topography and/or configuration conducive to receiving power.

In an embodiment, the other location includes, for example, another external device that includes, for example, at least one power transmitter or power receiver, and associated structures for at least one of using, storing, or re-transmitting power. A remote device for the lumen traveling device may also include power transmitters or power receivers.

In an embodiment, the lumen traveling device and system include control circuitry that may be part of the internal device components, and/or part of the system that is external to the lumen traveling device itself (e.g., a remote control or other computing device). In an embodiment, the control circuitry is implemented in logic forms (e.g., analog or digital logic circuitry and software, or both). In an embodiment, the control circuitry is stored as or implemented as non-transitory machine readable machinery. In an embodiment, data storage or usage can include implementation as non-transitory machine readable machinery.

In an embodiment, the lumen traveling device and system is configured for movement within a natural or artificial lumen of a subject's body. In an embodiment, the lumen traveling device and system is configured for passive movement; for example, the device is shaped to promote movement with natural flow or lumen movements, e.g., peristalsis. In an embodiment, the lumen traveling device and system is configured to utilize an external field, such as a magnetic field, to compel movement of the lumen traveling device, for example directly, as by a magnetic field exerting a force on the device, or indirectly, as in influencing an onboard controller. In an embodiment, the lumen traveling device and system is configured for active movement within a natural or artificial lumen of a subject's body and includes means for locomotion.

In an embodiment, the lumen traveling device may have a rolling motion, a crawling or walking motion (e.g., with leg-like protrusions), a swimming motion, an inchworm-like motion, a stick and slip motion, propelling motion, or a ciliated motion. In an embodiment, the lumen traveling device and system is configured for movement within a natural or artificial lumen of a subject's body according to direction provided by the controller in response to the one or more various sensors.

In an embodiment, the lumen traveling device includes as a means for locomotion, a propulsion system. In an embodiment the lumen traveling device includes a magnetohydrodynamic propulsion system that propels the lumen traveling device in a determined direction by ejecting a fluid jet. In an embodiment, the lumen traveling device includes an inertia-based propulsion system, for example an impulse-driven micromechanism having as a moving mass a permanent magnet that is driven by magnetic force achieved by applying a current to a coil. In an embodiment, the lumen traveling device includes a propeller. In an embodiment, a propeller can include a rotor driven by an electric or magnetic motor or actuator. In an embodiment, the lumen traveling device and system configured for movement according to direction includes a rudder, for example under control of a controller, to steer the device in a particular direction. In an embodiment, the lumen traveling device includes one or more appendages that function as paddles to propel the device, for example, through a fluid. In an embodiment, the lumen traveling device can include a linear actuator to drive paddle appendages in a manner so as to advance the device; combinations of paddles and their actuation can be used to induce movement in a particular direction. In an embodiment, the lumen traveling device can include internal permanent magnets configured to move a number of polymeric flaps or a single tail that provide thrust through the fluid. In an embodiment, the lumen traveling device can include a single tail of an electroactive polymer configured to provide thrust and direction.

In an embodiment, the lumen traveling device includes at least one locomotive mechanism configured to touch, grasp, grip, or otherwise engage the wall (e.g. surface) of a natural or artificial lumen of a subject's body. In an embodiment, the lumen traveling device includes at least one inchworm-like movement mechanism, in which at least a portion of the lumen traveling device intermittently engages and disengages from the wall of the lumen in a slip-and-stick fashion thereby traversing a distance. In an embodiment the lumen traveling device includes a vibratory locomotive mechanism, for example a mechanism inducing forced bending vibrations of continua of the lumen traveling device driven by actuators such as piezoelectric bending actuators. The locomotion direction of the lumen traveling device can be controlled by the excitation frequencies of the actuation element. In an embodiment, the lumen traveling device includes a sectional design, and each section is driven separately to engage or disengage the wall. In an embodiment, the lumen traveling device can include at least one actuator that drives the movement of the lumen traveling device and the engagement of the wall. In an embodiment, the lumen traveling device might include two-way linear actuators using a pair of springs made from a shape memory alloy. In an embodiment, the lumen traveling device might include a piezoelectric microactuator. In an embodiment, the lumen traveling device might include a micromotor. In an embodiment the lumen traveling device is jointed between sections of the lumen traveling device, and one or more actuators drive each section, for example, in a worm-like fashion. In an embodiment, the lumen traveling device includes an expandable bellow, for example, a pneumatic bellows that provides the locomotive mechanism. In an embodiment, the lumen traveling device includes surface-engaging protrusions, microprotrusions, adhesive micropilli, or clamps. In an embodiment, the lumen traveling device includes radially expandable portions that expand to engage and disengage the inner surface lumen of the lumen.

In an embodiment the lumen traveling device includes as a locomotive means an impelling mechanism configured to engage the wall (e.g., surface) and provide locomotion to the device; for example, an impelling device might comprise one or more appendages, legs, or wheels, with or without adhesive aspects such as micropilli. A number of mechanisms to actuate an impelling mechanism can be adapted for use with various embodiments described herein. In an embodiment, actuators and motors (micromotors) can be used to drive impelling devices. Examples of actuators include piezoelectric, DC motors, electromagnetic, and electrostatic actuators. In addition, actuators can be formed from shape memory alloys or ionic polymer metal components. In an embodiment, jointed appendages and legs can be actuated to propel the device forward in a walking or crawling motion.

As another example, a meso-scale legged locomotion system can include a slot-follower mechanism driven via lead screw to provide propulsive force to a jointed leg. In an embodiment, multiple jointed legs, e.g., of superelastic or other material, can be motivated to interact with the wall under control of a motor, e.g., a brushless minimotor. In an embodiment, appendages or legs can be formed from shape memory alloy and driven by the application of current. In an embodiment, appendages can act to engage the wall driven by rotational forces to provide locomotion. In an embodiment, wheels can be driven by motors or other actuators. In an embodiment, the lumen traveling device and system is configured to employ one or more impelling mechanisms in a manner to provide movement in a particular direction. In an embodiment, to change direction (e.g., as directed by a controller), only a portion of multiple appendages (or legs or wheels) can be actuated, thereby moving a portion of the device so that the device heads in a new direction and allowing the device to be steered.

In an embodiment, the lumen traveling device and system includes means for stabilization within the lumen, e.g., for maintaining orientation or position within the lumen. In an embodiment, the lumen traveling device and system includes one or more masses that can be steadied by an external field, for example a pair of permanent magnets that can be steadied in a magnetic field. In an embodiment, the lumen traveling device and system includes one or more gyroscope or one or more accelerometer. In an embodiment, the lumen traveling device and system includes one or more self-expanding stabilizing devices such as appendages, balloons, or capsules. A self-expanding stabilizing device can further have functionality in expanding the lumen.

In an embodiment, the lumen traveling device and system includes at least one location sensor to determine localization and spatial information regarding the lumen traveling device, including its position in three-dimensional (3D) space, the distance it has travelled along the lumen, and the region of the lumen in which it is located. A variety of technologies are known in the art to acquire such information, including but not limited to radio frequency (RF) triangulation, magnetic tracking, computer vision, and ultrasound. In an embodiment, the lumen traveling device and system includes an external device employing delivery of energy of one or more frequencies in the electromagnetic spectrum (e.g., radiowaves, microwaves, infrared, visible waves, ultraviolet waves, x rays, gamma rays) for tracking the lumen traveling device. In an embodiment, the lumen traveling device and system includes an imaging device (for example a magnetic resonance imager, x-ray imager, gamma camera, or the like) able to detect and track the lumen traveling device, which may be carrying a tag, for example a radiographic agent or contrast agent.

In an embodiment, the lumen traveling device and system includes a location sensor that is an ultrasound imaging device. In an embodiment an ultrasound imager housed in or otherwise associated with the externally alignable display and control device can be configured to utilize time of flight (ToF) between transmission of signals and reception of reflected signals to track the lumen traveling device, while the lumen traveling device is within the threshold of the location sensor. In an embodiment, the lumen traveling device is sensed when it is in the scanning plane, as determined by the location sensor. Alternatively or in addition, in an embodiment the lumen traveling device includes an ultrasound transducer that emits signals able to be received by one or more receivers, for example in the externally alignable display and control device or in an array of external receivers positioned on the body and in communication with the externally alignable display and control device.

In an embodiment, a method includes detecting at least one interaction of a lumen traveling device with a lumen of a subject by way of one or more sensors in or on a lumen traveling device; generating at least one sensed signal based on detection of the at least one interaction of the lumen traveling device with the lumen; determining if the sensed signal exceeds a threshold value for the at least one interaction; generating at least one communication signal based on the determination of whether the sensed signal exceeds the threshold value for the at least one interaction. In an embodiment, determining if the sensed signal exceeds a threshold value for the at least one interaction includes comparing the sensed signal to a reference data indicative of the threshold value. In an embodiment, the reference data is derived from at least one sensed signal, programmed by a user, or set while the device or system is in use. In this way, the interactions of the lumen traveling device with the lumen itself can be attributed more value, in that information is obtained from such interactions if a threshold is exceeded. For example, if the lumen traveling device is directed to sample the wall of the lumen, the lumen traveling device determines whether, for example, the sample size or location or type is sufficient to obtain the desired information. If such a threshold is exceeded, then the sample is taken and evaluated to provide the information sought. If the threshold is not satisfied, then the sample will not be taken at that time or location or in that manner, for example, and instead will be taken in another place, time, or manner, etc. so that the threshold evaluation can be conducted again.

In an embodiment, the lumen traveling device and system includes a location sensor that employs magnetic tracking of the lumen traveling device. In one example, the lumen traveling device includes at least one permanent magnet that is trackable by a magnetic sensor (e.g., magnetoresistive sensor) associated with (e.g., a skin-mounted array in communication with) or housed in the externally alignable display and control device. Alternatively or in addition, a magnetoresistive sensor inside the lumen traveling device can measure the intensity of the external magnetic field generated by external energized coils. In another example, for use with a lumen traveling device actively motivated by a low frequency magnetic field, a high frequency magnetic field can be used simultaneously for location purposes.

In an embodiment, the lumen traveling device and system includes a location sensor that employs inertial sensing to determine localization. For example, the lumen traveling device and system includes one or more accelerometers, which may function alone or in concert with an actuation field.

In an embodiment, the lumen traveling device and system includes a location sensor that utilizes radio frequency signals. In an embodiment, the lumen traveling device and system can include at least one external sensor array that evaluates an RF signal (e.g., for frequency and strength) transmitted by a transmitter housed in the lumen travelling device. The system can utilize information from the array to estimate distance and triangulate the signal. Approaches to RF signal-based localization methods include time-of-arrival (TOA), angle-of-arrival (AOA), time-difference-of-arrival (TDOA) and received-signal-strength (RSS) signal processing. In an embodiment, the lumen traveling device can include an RFID tag. In an embodiment, the lumen traveling device can include an RFID tag comprising a bidirectional, tridirectional, or omnidirectional antenna. In an embodiment, the lumen traveling device and system includes one or more software algorithms, e.g., to address signal propagation and reception, as well as noise reduction, can be used to increase efficiency and accuracy. In an embodiment, the lumen traveling device and system employs hardware and software to evaluate the phase difference of arrival at multiple frequencies of a signal to estimate the distance of the source to a receiver, together with linear least square estimation or other software algorithms.

In an embodiment, the lumen traveling device and system includes hardware and software to employ image comparisons to determine the position of the lumen traveling device. In an embodiment, images (e.g., moving picture experts group (MPEG)-7 images) are captured by the lumen traveling device. Images can be classified by hardware and software of the system utilizing, for example, vector quantization, principal component analysis, and neural networks, and/or event boundary detection algorithms, e.g., to identify topography, colors, elasticity, and the like.

In an embodiment, the lumen traveling device and system includes one or more location sensors that measure a distance. For example, a lumen traveling device can include a protrusion, (e.g., a wheel) attached to a counter (e.g., an odometer) that measures the distance the device has traveled, e.g., along a lumen wall. In an embodiment, a lumen traveling device can include a protrusion, (e.g., a flap) attached to a counter that measures the distance the device has traveled based on, for example the duration, force, or intermittent pulse, of pressure e.g., from fluid flow. See figures for more details.

In an embodiment, the lumen traveling device and system is configured for anchoring, at least temporarily, to the side wall of a lumen. In an embodiment, the lumen traveling device may include a wall-anchoring system with at least one of a hook, tether, peg, suction, spring, or adhesive. In an embodiment, the lumen traveling device includes at least one reservoir containing one or more adhesives. See figures for more details.

In an embodiment, the lumen traveling device is configured for easy removal from the lumen. In an embodiment the lumen traveling device is removable as a whole. In an embodiment, the lumen traveling device is removable in portions, e.g., after disintegration or degradation. In an embodiment, the lumen traveling device is configured for manual removal. In an embodiment, the lumen traveling device includes a tether, or other surface design, for removal through the introductory path. In an embodiment, a capsule endoscope used to image or treat the esophagus may include a tether for pulling the capsule back up through the mouth. In an embodiment, all or part of a lumen traveling device introduced into a lumen via a needle or catheter can be configured for manual removal via needle, catheter, etc., and may include magnetic or other attractive features. In an embodiment, all or part of the lumen traveling device is expelled via natural elimination. In an embodiment, a capsule endoscope traversing the gut can be expelled through the anus via natural digestive elimination. In an embodiment, all or part of a lumen traveling device can be expelled from a respiratory system via a cough. In an embodiment, all or part of a lumen traveling device having been introduced into a portion of a urogenital system, can be expelled via the urethra. In an embodiment, at the end of its life, biochemical remnants of a biodegradable lumen traveling device traveling in a blood stream can be eliminated via the liver.

In an embodiment, as described herein, at least part of the lumen traveling device is disposable. In an embodiment, as described herein, at least part of the lumen traveling device is biodegradable, so no retrieval from the subject is required.

In an embodiment the lumen traveling device includes at least one on-board instrument. In an embodiment the lumen traveling device includes one or more imaging devices. In an embodiment, the lumen traveling device can include a camera, a CCD sensor, a CMOS sensor, a spectroscopic camera (e.g., one that sees cells underneath the surface layer of tissue), or the like. In an embodiment the lumen traveling device includes one or more biopsy tool. In an embodiment, the lumen traveling device can include an aspiration tool, biopsy clip, biopsy punch, a curette, or the like. In an embodiment the lumen traveling device includes one or more deployment tool. In an embodiment, the lumen traveling device can include a mechanism for deploying a surgical clip or staple to a treatment site in the lumen (e.g., to a varix). In an embodiment, the lumen traveling device can include a mechanism for delivering a coil. In an embodiment the lumen traveling device includes a needle, for example to deliver a therapeutic agent directly to a treatment site on the lumen tissue. In an embodiment the lumen traveling device includes an energy emitter. In an embodiment, the lumen traveling device can include a wire that delivers heat to cauterize a tissue. In an embodiment, the lumen traveling device can include a thermal tool for ablating a tissue. In an embodiment, the lumen traveling device can include an ultrasound emitter or the like.

In an embodiment, the lumen traveling device includes at least one sampling means, as described herein. In an embodiment, the lumen traveling device includes a liquid capture device, for example a reservoir or adsorbant or absorbant material. In an embodiment, sampling means are housed in leg-like protrusions that engage the lumen wall (e.g., nano or micro calipers 375 configured to grasp cells or DNA of the lumen, suction cup feet-like bases 385 that include nano or micro teeth, bristles, or needles for sampling cells or DNA of the lumen). In an embodiment, the sampling means is able to obtain small samples of blood, tissue, cells (including, for example, microorganisms or components thereof), nucleic acids, proteins, etc. from the lumen of the subject.

In an embodiment, the lumen traveling device and system is configured to image or map a lumen. In an embodiment, the lumen traveling device and system is configured to provide treatment in a lumen. In an embodiment, the lumen traveling device is followed in the lumen by the externally alignable display and control device (EADCD) in real-time and spatial alignment. In an embodiment, the lumen traveling device can be directed to advance or return to a site in the lumen by the externally alignable display and control device in real-time, and further action by the lumen traveling device can be directed via the externally alignable display and control device. In an embodiment, the lumen traveling device and system is used to image, map, or provide treatment to a lumen that includes at least a portion of an alimentary canal, a blood vessel, a respiratory tract, a urinary tract, genital tract, a duct of an organ, or the like.

In an embodiment, a system includes a memory device wherein the memory device is configured to retrieve data associated with a specific location corresponding to a lumen reference path previously traveled by the lumen traveling device when queried. In an embodiment, the data is not retrievable unless the EADCD is within a proximity threshold from where the LTD actually traveled in the lumen (e.g., the proximity threshold includes at least one of approximately one millimeter, approximately ten millimeters, approximately 100 millimeters, approximately one centimeter, or approximately ten centimeters from the actual path traveled in the lumen. In this way, the reference map is a predetermined pathway intended for the LTD to follow, while the reference path is a map of the actual path the LTD took as it traveled through a lumen. Thus, the reference path may not ideally follow the exact reference map of the lumen, but should be approximately the same. In an embodiment, the LTD does not transmit data unless and until it enters a threshold range of a predetermined location (e.g., a specific location in the lumen such as a specific section of the intestinal tract of a subject). In this way, the LTD conserves power and can be manufactured with lightweight, thin battery power source. In an embodiment, the LTD is programmable to not transmit data until it reaches the predetermined target location. In an embodiment, the LTD is remote controllable to not transmit data until it reaches the predetermined target location. For example, the LTD emits a location beacon or signal to verify its location as it travels through the lumen, only transmitting additional data about the condition of the lumen (e.g., biological tissue sampling, therapeutic agent delivery, etc.) until it reaches the predetermined target location.

For example, the lumen traveling device can be introduced into any portion of an alimentary canal, such as the esophagus, stomach, small intestine, large intestine, and the like, through ingestion or delivery (e.g., by conventional endoscope or suppository). For example, the lumen traveling device can be used to image some or all of the alimentary canal to look for anomalies such as but not limited to polyps, tumors, varices, bleeding, obstructions, inflammation, and the like. In an embodiment, the lumen traveling device can be used to perform a treatment in the alimentary canal, such as treatment of a gastrointestinal bleed by delivering energy (e.g., thermal energy as in cauterizing or freezing or radiofrequency ablation) or by delivering a ligature (e.g., clip or band) or by injecting a compound (e.g., cyanoacrylate or epinephrine). In an embodiment, the lumen traveling device is followed in the alimentary canal by the externally alignable display and control device in real-time and spatial alignment. In an embodiment, the lumen traveling device can be directed to advance or return to a site in the alimentary via the externally alignable display and control device in real-time using spatial alignment, and further action by the lumen traveling device as described above can be directed via the externally alignable display and control device.

As described herein, the alignment of the EADCD can optionally first be aligned with external markers (e.g., based on a fiducial sensor, etc.) and further aligned with the LTD (e.g., based on the LTD sensors) to retrieve data associated with the location of the LTD or the reference path.

In an embodiment, the lumen traveling device is used to image, map, or provide treatment to a lumen that is a blood vessel or lymphatic duct. In an embodiment, the lumen traveling device can be injected into a blood vessel and used to image the blood vessel for the presence of, e.g., plaque, stricture, or stenosis, and if necessary to provide treatment by delivering an expandable stent to the area. In an embodiment, the lumen traveling device used to image a blood vessel for the presence of an embolism and deliver an agent for degrading the embolism. In an embodiment, the lumen traveling device can be used to image a blood vessel for the presence of an aneurysm and if necessary provide treatment by delivering a clip or coil to the site. In an embodiment, the lumen traveling device can be followed in the blood vessel by the externally alignable display and control device in real time.

In an embodiment, the lumen traveling device can be directed to advance or return to a site in the blood vessel by the externally alignable display and control device in real time, and further action by the lumen traveling device as described above can be directed via the externally alignable display and control device. In an embodiment, a lumen traveling device is injected into a blood vessel in the lower extremity of a subject experiencing pain and poor healing in the limb. By alignment and movement as described herein, the externally alignable display and control device is used to direct the lumen traveling device into several branches of the blood vessel while displaying the results in real time until an area of stenosis is detected. The externally alignable display and control device is then used to direct the lumen traveling device to deploy an expandable stent utilizing extended appendages to expand the stent to fit the vessel. The externally alignable display and control device is then used to direct the lumen traveling device back to the site of entry by moving the externally alignable display and control device over the limb while viewing the progress of the lumen traveling device in real time, and the lumen traveling device is retrieved via a syringe. Similarly, a lymphatic duct can be imaged or treated in the same manner as a blood vessel. In an embodiment, inflammation (e.g., associated with cancer or infection, etc.) can be monitored by a lumen traveling device deployed in the lymph system or the vasculature.

In an embodiment, the lumen traveling device and system is used to image, map, assist in diagnosis, sample, or provide treatment to a lumen that is part of a urinary tract. In an embodiment, a lumen traveling device can be introduced into a urinary tract via the urethra (e.g., by catheter and/or locomotive aspects described herein) and used to image the urethra, bladder, ureters, and kidney ducts for the presence of, In an embodiment, tumors, strictures, bleeding, ulcers, stones, inflammation, infection, or the like. In addition, the lumen traveling device can be used to perform a treatment in a urinary tract such as disintegration of a stone, biopsy or removal of a tumor, directed killing of a microorganism or the like. In an embodiment, the lumen traveling device is followed spatially in real time in the urinary tract by aligning the externally alignable display and control device. In an embodiment, the lumen traveling device can be directed via the externally alignable display and control device in real time to advance (e.g., to ensure the entire bladder has been viewed) or to return to a previously viewed site in the bladder (e.g., by moving the externally alignable display and control device across the external abdomen), and further action by the lumen traveling device as described above can be directed via the externally alignable display and control device.

In an embodiment, a lumen traveling device can be introduced into a male reproductive system via the urethra (e.g., by catheter and/or locomotive aspects described herein) to image or treat a site therein. In an embodiment, the lumen traveling device is followed in the male reproductive system by the externally alignable display and control device in real time. In an embodiment, the lumen traveling device can be directed spatially via the externally alignable display and control device in real time to advance or to return to a site, and further action by the lumen traveling device as described above can be directed via the externally alignable display and control device. In an embodiment, a lumen traveling device is directed by the externally alignable display and control device to advance to a site in the vas deferens of a subject who has undergone a past vasectomy to evaluate the efficacy of the vasectomy procedure. If the vas deferens is not fully occluded, the lumen traveling device is directed to deliver a clip to completely block the lumen.

In an embodiment, a lumen traveling device can be introduced into a female reproductive system via the vagina (e.g., by direct delivery and/or locomotive aspects described herein) and used to image the vagina, cervix, uterus, and fallopian tubes for the presence of, for example, tumors, genital warts, strictures, tubal pregnancy, tubal ligation, abnormal bleeding, endometriosis, ulcers, inflammation, infection, or the like. In addition, the lumen traveling device can be used to perform a treatment in a reproductive tract such as ablation of tissue, biopsy or removal of a tumor, directed killing of a microorganism or the like. In an embodiment, the lumen traveling device is followed in the reproductive tract by the externally alignable display and control device in real time. In an embodiment, the lumen traveling device can be directed via the externally alignable display and control device in real time to advance (e.g., to ensure the entire uterus has been viewed) or to return to a previously viewed site in the reproductive tract, and further action by the lumen traveling device as described above can be directed via the externally alignable display and control device. In an embodiment, the lumen traveling device is directed via the externally alignable display and control device to the fallopian tube to evaluate the presence of endometrial tissue occluding the fallopian tube and potentially preventing pregnancy. If occlusion is identified, the externally alignable display and control device is used to direct the lumen traveling device to emit thermal energy to ablate the tissue and open the tube. In an embodiment, the externally alignable display and control device is then used to direct the lumen traveling device to the other fallopian tube by moving the externally alignable display and control device over the external abdomen while viewing the progress of the lumen traveling device in real time.

In an embodiment, the externally alignable display and control device includes at least one projector or display. In an embodiment, the externally alignable display and control device includes a projector configured to project at least one hologram (e.g., on the subject's body, on a surface, or into air).

In an embodiment, the externally alignable display and control device (EADCD) includes a liquid crystal display (LCD), light-emitting diode display (LED), or a projection display. In an embodiment, the EADCD includes an organic light emitting diode (OLED) or similar device that includes a sterile surface, and sufficient flexibility to function despite folds or creases. In an embodiment, an organic light emitting diode includes an anode, cathode, OLED organic material, and a conductive layer. In an embodiment, the OLED includes a double layer structure with separate hole transporting and electron-transporting layers, with light emission sandwiched in between the two layers. In an embodiment, the EADCD includes multiple distinct display units forming one or more larger displays, with each display unit informed and controlled by the processor and controller, which may be indicating the sensed signals from the sensors. In an embodiment, the EADCD may include a flexible backing, e.g., a rubber polymer, with discrete rigid display units (LCD, LED, or OLED, for example). In an embodiment, information is displayed through multiple distinct display units (e.g., having LCD, LED, or OLED technology) combining to form an EADCD configured to provide displayed information; which information is displayed on which unit is determined optionally in real-time by the processor and controller using signals provided by sensors determining the location of the EADCD on the subject's body and the location of the lumen traveling device inside the subject's body, and optionally the location of each relative to the other. In an embodiment, the EADCD is flexible, foldable, or otherwise able to be rearranged (e.g., a foldable OLED display). In an embodiment, the EADCD includes at least one projector.

In an embodiment, a polymer light emitting diode (PLED) can be utilized, since it emits light under an applied electric current. Typically, a PLED utilizes less energy than an OLED to produce the same level of luminescence. In an embodiment, the PLED includes at least one of a derivative of poly(p-phenylene vinylene) and polyfluorene. In this example, the light comes from a single layer of electroluminescent polymer, which is held between two transparent elastic composite electrode layers.

In an embodiment, the EADCD includes a flexible or stretchable display including intrinsically stretchable OLEDs formed by elastic constituent materials, for example carbon nanotube (CNT)—polymer composite electrodes sandwiching an electroluminescent polymer blend layer or an elastic electroluminescent blend with an ultrathin gold coating on polydimethylsiloxane substrate and gallium—indium eutectic alloy liquid metal as the opposite electrode. In an embodiment, the EADCD includes a flexible or stretchable display comprising intrinsically stretchable PLEDs including an electroluminescent polymer layer sandwiched between a pair of transparent elastomeric composite electrodes based on a thin silver nanowire (AgNW) network. In an embodiment, the EADCD can provide real-time display of information by utilizing specific pixels of a flexible display and combining them to form a cohesive image, as controlled by the processor and controller and informed by sensors detecting the lumen traveling device, or at least one physiological characteristic of the subject. In an embodiment, noncontiguous portions of a display may be utilized (e.g., light-emitting diodes emitting light) in such a manner as to complete an image.

In an embodiment, the EADCD includes an organic light emitting device (OLED). In an embodiment, the EADCD includes a flexible organic light emitting diode (FOLED) that incorporates a flexible plastic substrate on which the electroluminescent organic semiconductor is deposited. In an embodiment, the EADCD includes other illumination devices, such as silicon LEDs, LCD, electro-luminescent devices, incandescent, or chemical devices.

In an embodiment, the EADCD includes a flexible electronic paper based display. In an embodiment, the dynamic display includes a plastic flexible display with an organic thin film transistor (OTFT).

In an embodiment, the EADCD includes a dedicated device (e.g., a device held, for example, between the thumb and forefinger of the subject itself or a healthcare provider). In an embodiment, the dedicated device is sized and shaped like a cell phone, or tablet. In an embodiment, the EADCD includes a cell phone or tablet itself. In an embodiment, the EADCD includes a user interface, and circuitry configured for running at least one computer program for monitoring the LTD. In an embodiment, the dedicated device is sized and shaped to be worn on a hand (e.g., a device worn like a glove, watch, bracelet, badge, etc.). In an embodiment, the dedicated device is sized and shaped to be worn on one or more fingers (e.g., a device worn as a ring).

In an embodiment, as described herein elsewhere, the EADCD includes at least one inertial sensor, accelerometer, proximity sensor, or landmark reader or fiducial reader (e.g., RFID, laser, etc.). In an embodiment, the EADCD includes at least one topography sensor for detecting landmarks on a skin surface (e.g., an imaging sensor, optical sensor, etc.). In an embodiment, the system further includes means to align the externally alignable display and control device with the path previously traveled by the lumen traveling device, including at least one of at least one inertial sensor, at least one fiducial sensor, at least one topographical sensor, or at least one laser pointer. In an embodiment, the topographical sensor includes at least one of an imaging sensor, or optical sensor. In an embodiment, the at least one fiducial sensor includes at least one optical sensor, radiographic sensor, radiofrequency sensor, or magnetic sensor.

In one example, the EADCD detects the topography of the skin area (e.g., by scanning the rough surface of the skin) as the lumen traveling device and system makes a first pass at imaging a site in the underlying lumen and records the results in memory, then on a subsequent pass, the EADCD scans the skin again and using comparison to the original scan identifies the site, then controls the LTD to the site. In another example, the EADCD uses triangulation between fiducials in the body (e.g., surgical staples) or on the body (e.g., placed on the skin at the beginning of the procedure) to align with a body site, then the EADCD controls the LTD to the corresponding site in the lumen.

In an embodiment, the LTD includes at least one wired or wireless connection between the one or more sensors (or sensor assemblies) and the EADCD, by way of a processor and/or controller. For example, in an embodiment, the electronic circuitry receives information from the one or more sensors or sensor assemblies and determines if, for example, the LTD should change speed, direction, or release a tag or therapeutic agent, or take a biological sample of the lumen, and informs the controller.

In an embodiment, the processor can be programmed to select a particular location along the lumen to sample or treat by release of a therapeutic agent, or to tag for further analysis, or can be directed by the user (e.g., by a user interface), where the user includes the subject itself, a healthcare worker, a computer, or other user. Thus, the controller is configured to adjust the function of the LTD and/or EADCD including their function relative to each other. In an embodiment, a processor can be configured to receive at least one signal from the one or more sensors or sensor assemblies regarding at least one of location of the LTD, the status of any biological sampling obtained or scheduled to be obtained, the release of any therapeutic agents or schedule of release based on what was put on board before the LTD began the lumen travel path.

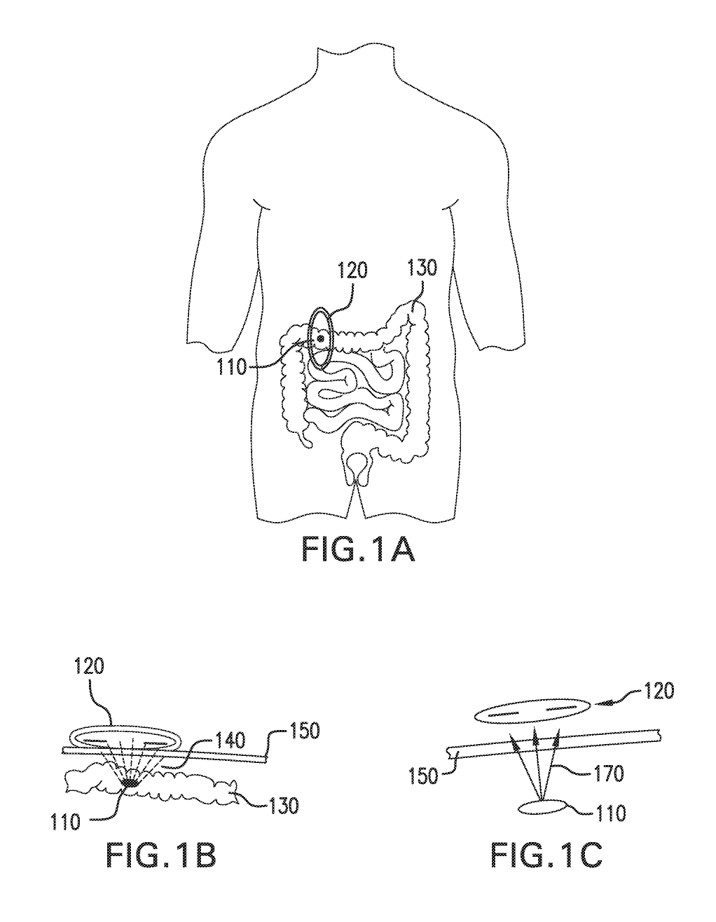

Turning to the Figures, as shown in FIG. 1A, in an embodiment, a system 100 includes a LTD 110 traveling through a lumen 130 (e.g., intestinal tract), the LTD being sensed when the EADCD 120 is placed in planar proximity to the LTD that is located internal to the subject. As shown in FIG. 1B, the EADCD 120 and LTD 110 are able to cross-talk through the body surface of the subject when the EADCD is placed in planar proximity to the LTD, even though the LTD is located in a lumen 130 of the subject. In an embodiment, the LTD is configured to transmit 140 (e.g., RF transmission) through the lumen 130 surface and through the subject's skin 150, to the EADCD 120 that is external to the subject's body. In an embodiment, as set forth in FIG. 1C, the LTD 110 is able to transmit image data 170 (e.g., RF transmission) to the EADCD 120 through the skin 150 of the subject.

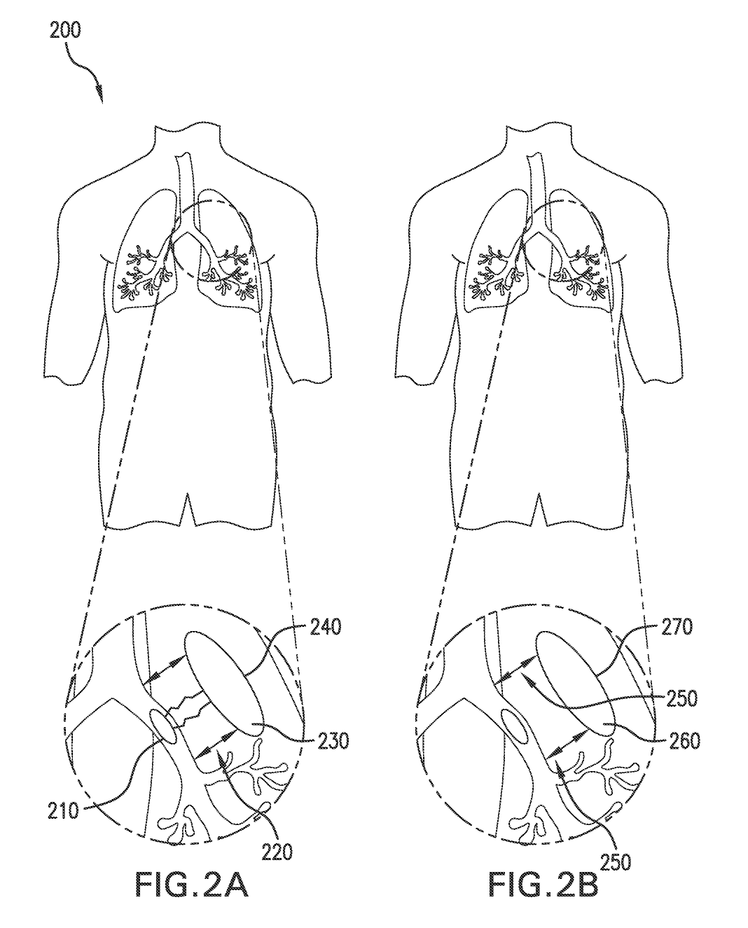

As shown in FIG. 2A, in an embodiment, the LTD 210 is configured to transmit 220 (e.g., NIR transmission) images to the EADCD 230 in real time from a lumen. In an embodiment, the real-time lumen image 240 is projected or otherwise displayed externally to the subject's body. As shown in FIG. 2B, the EADCD 270 is passed over a location of the subject's body that is marked 250 (e.g. by fiducials or sensors, including NIR location sensors, etc.) and is planar to the travel path of the LTD (including a historical travel path, the location of which is stored data), and the stored lumen image 260 is projected or otherwise displayed externally to the subject's body.

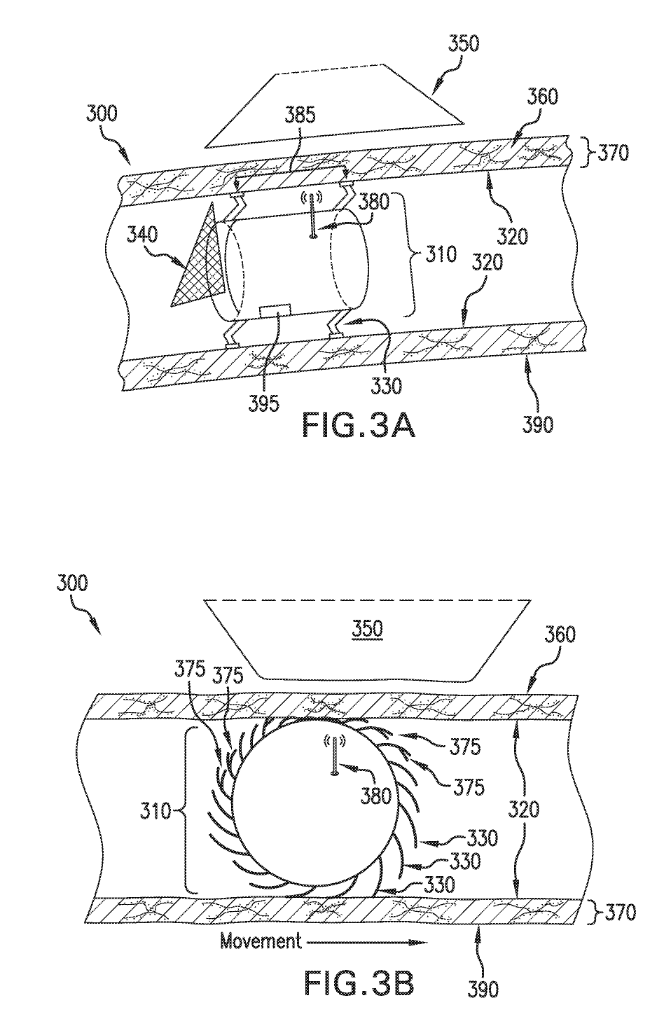

As shown in FIG. 3A, in an embodiment, the system 300 includes a LTD 310 that travels through a lumen by propelling itself with a paddle or rudder 340, and/or leg-like protrusions 330, which have optional suction cup bases 385 configured for biological sampling of the lumen wall 320. In an embodiment, the LTD 310 is in wireless communication 380 with the EADCD 350 outside of the subject's body. Communications between the LTD 310 and EADCD 350 can occur through the skin 370 of the subject, when the EADCD 350 is aligned planar to the LTD 310 and can include contact with the outer surface 360 of the subject's skin. In an embodiment, one or more biological sampling base 385 can be configured to sample at various depths of the lumen wall, including outward toward the skin 360 or inward toward the inner wall 390 of the lumen. In an embodiment, a power source 395 is included in the LTD 310. In an embodiment (not shown) at least one therapeutic agent compartment is contained in the LTD and configured for release of the at least one therapeutic agent as the LTD moves through the lumen. As described herein, the release can be programmed to be at a specific location along the lumen, or at multiple scheduled time points or locations. In an embodiment, the release is gradually along at least part of the lumen traveling path.

In an embodiment, if an anti-inflammatory or coagulant is desired along the lumen pathway, one or more can be loaded into the LTD prior to deployment into the lumen, and subsequently can be remotely directed by the user to release the one or more agents at specific locations, or can be directed by computer program. In an embodiment, the therapeutic agent includes, but is not limited to, an anti-inflammatory agent, coagulant, anti-coagulant, anesthetic, analgesic, vitamin, mineral, chemotherapy agent, antibiotic, antimicrobial (e.g., antibiotic, antifungal, antiparasitic, or antiviral agent), vascular dilator, vascular constrictor, hormone, steroid, cytokine, chemokine, muscle relaxant, anti-spasmodic.

In an embodiment (not shown) at least one onboard instrument is contained in the LTD and configured for use as the LTD moves through the lumen or at a particular site along the lumen. In an embodiment, the at least one onboard instrument includes, but is not limited to an imaging device, a biopsy tool, a deployment tool (e.g., for deploying a surgical clip or staple), a needle, or an energy emitter.

As shown in FIG. 3B, in an embodiment, a system 300 includes a LTD 310 moving in the direction of the arrow, through the lumen by contacting the inner walls of the lumen 320 with the various leg-like protrusions 330, some of which include caliper-like sampling devices 375 for biological sampling of the lumen. In an embodiment, the caliper-like sampling devices 375 are configured to access the lumen wall 370 to the inner part of the subject's body 390 or outward toward the skin 360. In an embodiment, the LTD 310 is in wireless communication 380 with the EADCD 350 that is outside of the subject's body, and optionally contacting the skin 360 in a planar location relative to the LTD 310. As indicated elsewhere herein, the LTD 310 can take various forms and shapes without losing the character of the structure or function of the device as described herein, even though not all possible combinations are illustrated.

In an embodiment, a method, system, device, and/or computer program product relate to various embodiments disclosed herein.

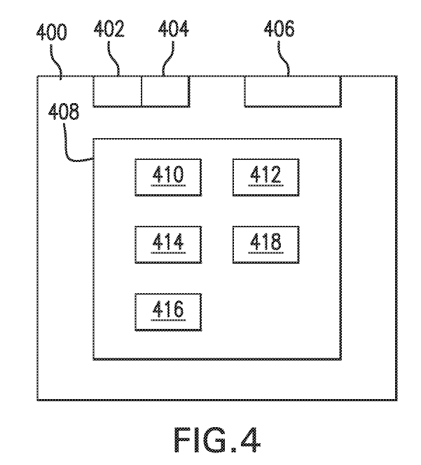

As illustrated in FIG. 4, a diagram of an example of a processing circuit 400 for completing various embodiments of the systems and methods disclosed herein is shown. In an embodiment, the processing circuit 400 is generally configured to accept input 402 from at least one sensor. The processing circuit 400 can be configured to receive configuration and reference data 412. Input 420 data can be accepted continuously or periodically. The processing circuit 400 analyzes data provided by one or more sensors, to determine the next action for the LTD, and instruct the controller (not shown). Based on the detected parameters as described herein, the processing circuit 400 may notify the EADCD, another external computer or computing system, or an on-board computing component to execute the next action. The processing circuit 400 can also generate real-time or updated maps of the lumen in which it is traveling, or can instruct the LTD to stop, hover, attach to the lumen, change direction, release a therapeutic agent or tag, etc. In determining the analysis, the processing circuit 400 can make use of machine learning, artificial intelligence, interactions with databases (including reference data), pattern recognition, logging, intelligent control, fuzzy logic, neural networks, etc.

In an embodiment, the processing circuit 400 includes a processor 406, which can be a specific use computer in certain instances. In an embodiment, the processor 406 is part of a general use computer. In an embodiment, an application of specific integrated circuit (ASIC), one or more field programmable gate arrays (FPGAs), digital-signal-processor (DSP), group processing is included. In an embodiment, the processing circuit 400 includes memory 408. In an embodiment, memory 408 is one or more devices (e.g., RAM, ROM, Flash memory, hard disk storage, etc.) for storing data and/or computer code for facilitating the various processes described herein. Memory 408 may be included as non-transient volatile memory or non-volatile memory. In an embodiment, memory 408 includes at least one of database components, object code components, script components, or other information structure for supporting the various activities and information structures described herein. In an embodiment, memory 408 can be communicably connected to the processor 406 and can include computer code or instructions to the controller (not shown) for executing the processes described herein.

In an embodiment, the memory 408 includes memory buffer 410 configured to receive data from one or more sensors via input 402, and includes, for example, a real-time data stream from one or more sensors. In an embodiment, the data received via the input 402 can be stored in memory buffer 410 until it is accessed by various modules of the memory 408, including a sensor module 414 or feedback module 416. In an embodiment, the memory 408 includes configuration data 418 and can include, for example, information related to engaging with other components (e.g., sensors of the system, the LTD itself, the EADCD, etc.) and can include a command set for interfacing with a computer system used to transfer user settings or otherwise set up the system (e.g., graphical user interface controls, menus, visual information, etc.). In an embodiment, the configuration data 418 can include a command set needed to interface with communication components (e.g., a universal serial bus (USB) interface, Wi-Fi interface, Ethernet, etc.). In an embodiment, the processing circuit 400 can format data for output 404 to allow a user to configure the system as described herein. The processing circuit 400 can also generate commands needed to generate visual or audio warnings for display on the EADCD, or a speaker thereof. In an embodiment, the processing circuit 400 also generates commands needed to drive haptic or other mechanical feedback (e.g., vibration). In an embodiment, the configuration data 418 can include information as to how often input should be accepted from a sensor or determine the default values required to initiate communication with sensors or other components of the processing circuit 400 or other systems described herein.

In an embodiment, the processing circuit 400 further includes output 404 configured to provide output to the EADCD or another output device, or components of the system as described herein. In an embodiment, the feedback module 416 generates feedback to produce output via a feedback device (e.g., EADCD), including output as information to a display, audio speaker, haptic response, or network signal. As described herein, in an embodiment, a non-transitory computer-readable medium having instructions stored thereon, the instructions forming a program executable by a processing circuit to instruct the LTD of a next action as disclosed herein.

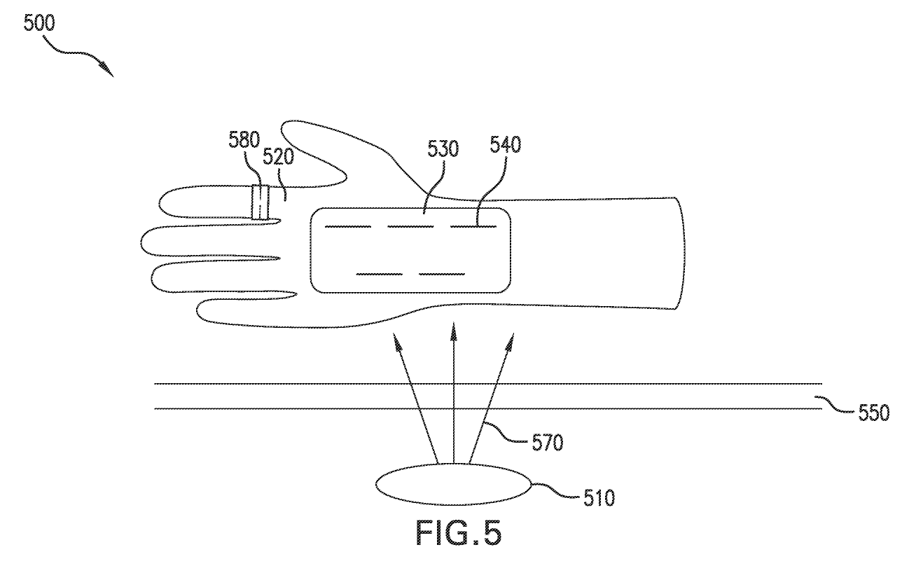

As disclosed in FIG. 5, the system 500 includes a lumen traveling device 510 is able to transmit image data 570 (e.g., RF transmission) to the EADCD 530, 580 as part of a glove 520 or ring 580 through the skin 550 of the subject. As indicated, the EADCD includes a display 540 of transmitted data 570 from the lumen traveling device 510.

### Prophetic Example 1

### A Lumen Imaging System is Used to Visualize and Localize Intestinal Lesions, Polyps, and Tumors in Real Time

A lumen imaging system includes a camera-bearing lumen traveling device (LTD) and a handheld externally alignable display and control device (EADCD). The EADCD functions like a computer interface having input (e.g., computer mouse or graphic interface) and output (e.g., display) capabilities. Intraluminal images, transmitted by the LTD, are received and displayed by the handheld externally alignable display and control device in real time, but only when the EADCD is aligned directly over the LTD. Real time display of images transmitted by the LTD from an intraluminal location provides immediate feedback for a physician, while wireless input functions allow the physician to use the EADCD to direct the LTD to areas of interest or to reexamine selected areas of the intestine. Records of the displayed images and their corresponding locations in the gastrointestinal tract are stored in the display device. The lumen imaging system allows control of the LTD using the EADCD in a way that is analogous to control of a screen cursor by a computer mouse. Additionally, input capabilities on the EADCD, including a graphical interface and touch screen, permit two-way communication with the LTD as well as access to internal storage for retrieval of images previously obtained at specific locations

The LTD, which is an ingestible capsule approximately 11 mm by 26, mm includes cameras, light sources, transmitters, receivers, control circuitry, memory, location sensors, a battery, and a means of locomotion to move within the gastrointestinal tract. The cameras, located at each end of the LTD, include a complementary metal oxide semiconductor (CMOS) image sensor and an adjustable lens. Each lens is surrounded by light emitting diodes (LEDs) to illuminate the intestinal wall. For example, a micro-camera with a 0.6 mm color lens with magnetic coils for focus adjustment, a CMOS image sensor, and 4 white LEDs can be adapted for use with the instant embodiment. Application-specific integrated circuitry is designed to process and transmit image data, and to receive and act on command signals from the display device. For example, a transceiver chip capable of transmitting image data at 20 Mbps on a 500 MHz RF channel has been used with locating devices in the gastrointestinal system of patients, and can be adapted for use with this embodiment described herein. Control circuitry to actuate the cameras, the location sensors, and the locomotion system is included with memory to allow programming of the LTD.

Sensors are incorporated in the LTD to identify the location of the device. For example, image analyzers are used to identify intestinal locations (e.g., duodenum, ileocecal valve, cecum), or lesions, polyps, tumors or inflammation sites, and record the locations in memory. Additional location sensors may include a pH sensor that determines pH in the intestine or a time-keeping device that records the elapsed time of transit for the LTD. Intraluminal images transmitted in real time to the display device are informationally linked to the coincident location identifiers. For example, serial images of inflammation in the small intestine are coded so as to be linked to the corresponding elapsed times of transit for the LTD. Momentary display of interesting intestinal images (e.g., images showing inflammation of the small intestine) may indicate a need for reexamination or exploration of the inflamed region. Query of the LTD by the EADCD for location identifiers (e.g., elapsed transit time, image analyses, and pH results) associated with the display of the inflammation region, and subsequent instructions to the LTD enable the physician to direct the return of the LTD to the site for further analysis.

The EADCD is used to control movement of the LTD in the region of the intestine. The LTD has a locomotion system and position control circuitry that responds to signals from the EADCD. The locomotion system includes approximately 1, 2, 3 or more legs which permit travel through the intestine by alternately bracing against the intestinal wall and extending in the direction of travel. For example, a device with jointed legs that is mobile in tubes and channels containing bends and obstructions can be adapted for use with the instant embodiment in a lumen of a patient. For example, capsule endoscopic devices with multiple legs for use in a digestive system have been described and can be adapted for use with the instant embodiment in a lumen of a patient. The articulated legs are moved by leg controls that include circuitry and motors to actuate the legs in response to signals from the physician (or system operator) that are relayed by the display device. Motion control circuitry connecting sensors and locomotion mechanisms for micro-robots can be adapted for use with the instant embodiment. Movements and associated images captured by the LTD are informationally linked to their location in the intestine as identified by image recognition (intestinal landmarks), pH, time of travel, or other location identifiers. Movement of the LTD is controlled by a physician/operator using the EADCD. Real time imaging informs the operator as to which direction to move the LTD, and movement of the EADCD sends signals to actuate the articulated legs on the LTD. Further external systems to control the motion of capsule endoscopes can be adapted for use with the instant embodiment.

The externally alignable display and control device (EADCD) receives images transmitted by the LTD. Image data transmitted at radio frequencies is received by transceivers in the display device, but the signal is filtered to only allow receipt of transmissions emanating from immediately under the EADCD (see FIG. 1A-C). Computational methods to filter signals and localize medical devices in the digestive tract can be adapted for various embodiments described herein. In an embodiment, location-dependent signal parameters including: angle of arrival, time of arrival and received signal strength are used to estimate the location of a transmitting device and may be used to filter the signals received from the LTD. In this embodiment, images will be displayed by the EADCD only when the EADCD is directly over the LTD.

The image data and corresponding location data is stored in memory on the LTD until the LTD passes out of the intestinal tract of the patient. A temperature sensor in the LTD monitors the ambient temperature and signals a control circuit when the temperature falls below body temperature after the LTD exits the anus. The control circuit provides current to the memory units on the device, erasing any imaging, location and patient identification data. Temperature controls for computer memory comprising temperature sensors and resistors have been described and can be adapted for use with the instant embodiment.

### Prophetic Example 2

### A Lumen Imaging System with Location-Specific Display of Stored Intraluminal Images is Used to Monitor Lung Cancer

An intraluminal imaging system includes a lumen traveling device (LTD) and an externally alignable display and control device (EADCD) capable of recalling images obtained at specific lumen locations. To monitor lung cancer, initial intraluminal images of lung cancer tumors are obtained with a LTD introduced into the bronchial tree by inhalation or bronchial scope. Images of any tumors and their locations in the airway are transmitted to the EADCD and stored in memory. In several weeks, following chemotherapy, the LTD is reintroduced in the airway, and the system is used to repeat imaging of the tumor locations. The current images are compared to the pre-chemotherapy images. The LTD locomotion system and real time image display and location sensing are used to guide movement of the lumen traveling device using a handheld EADCD.

The LTD is fabricated from biodegradable components, such as silk or paper. The lifespan of the device can be altered based on the crystalline structure of the silk, which dictates the rate at which water accesses the silk structure and degrades it. In this way, the device can be designed for a lifespan of minutes, days, months, or even years. Likewise, magnesium or silicone can also be utilized, based on the specific design parameters desired for a biodegradable device.

A patient with suspected lung cancer nodules is imaged with a lumen imaging system using a LTD introduced into the airway and an EADCD to display the localized images in real time and to control the movement of the LTD. The capsular LTD is approximately 7 mm in diameter and 23 mm in length and includes: a camera, a light source, transmitters, receivers, control circuitry, memory, location sensors, a battery, and a means of locomotion. A high resolution camera, responsive to external signals, includes a CMOS image sensor, an adjustable lens and circuitry to process and transmit image data in real time. For example, a micro-camera with a 0.6 mm color lens with magnetic coils for focus adjustment; a CMOS image sensor, and 4 white LEDs can be adapted for specific embodiments. For example, imaging devices, high performance electronics, and radio frequency electronics formed from bioresorbable materials can be adapted for various embodiments. Intraluminal images transmitted by the LTD are only received and transmitted by the EADCD when the EADCD is immediately over the LTD. See FIG. 1.

Methods to filter signals and localize medical devices in the digestive tract can be adapted for use with the instant embodiment. For example, location-dependent signal parameters including: angle of arrival, time of arrival and received signal strength are used to estimate the location of a transmitting device and may be used to filter the signals received from the LTD. Image data transmitted from the airway by the LTD are received by the EADCD and stored in memory along with linked location sensing data.

The EADCD is a handheld device (e.g., smart phone) with transceivers, display capability and location sensors to display intraluminal images in real time and remember their location in the body. Image data transmitted at radio frequencies is received by transceivers in the EADCD, but the signals are filtered to only allow receipt of transmissions emanating from immediately under the EADCD (see FIG. 1C). Computational methods to filter signals and localize medical devices in the digestive tract are described and can be adapted for use with the instant embodiment. In an embodiment, location-dependent signal parameters including angle of arrival, time of arrival and received signal strength are used to estimate the location of a transmitting device, and may be used to filter the signals received from the LTD. In this embodiment, images will be displayed by the EADCD only when the EADCD is directly over the LTD.

The EADCD has location sensors to identify body locations at the time images are displayed. In an embodiment, the EADCD may have near infrared (NIR) sensors to detect landmark subsurface features in the lungs, such as vasculature patterns, or patterns of blood within vasculature that act as markers to identify a location in the lung. See FIG. 2A.

Methods and systems to obtain and store landmark features can be adapted for use with the instant embodiment, for example images of landmark features (location identifiers) are linked to simultaneously transmitted intraluminal images from the LTD. Registration circuitry on the EADCD identifies landmark features previously stored in memory and recalls the linked intraluminal images. In an embodiment, passing the handheld EADCD over the location of a lung tumor previously imaged with a LTD will recall the LTD image linked to the landmark feature (e.g., vascular pattern at the tumor site). See FIG. 2B.

Multiple intraluminal airway images and their associated landmarks may be accessed by moving the EADCD with NIR sensors across the body surface. Moreover, initial intraluminal images may be compared to images obtained at a later time. In an embodiment, intraluminal imaging with a LTD is done before and after treatment of a lung tumor with chemotherapy. Revisiting the same sites in the airway is guided by the stored landmark features. Registration of the EADCD and the LTD with the landmark features at the tumor site allows comparison of the images obtained before and after chemotherapy.

Steering and positioning of the LTD inside the airway lumen is directed by the physician, caregiver, or patient himself, using the handheld EADCD. The EADCD directs a magnetic field to move and position the LTD within the airway lumen. The LTD contains magnetic components which are responsive to an externally applied magnetic field. If the device is designed to be biodegradable, detachable magnetic iron filings or legs can be included. In an embodiment, the iron filings are coated in silicone and the entirety is biodegradable. In an embodiment, the detachable magnetic components are not biodegradable but are retrievable by a magnet or endoscope, or are naturally expelled by cough once the remainder of the device has biodegraded.

The EADCD may direct varying magnetic fields to move the LTD. For example, magnetic steering and positioning systems for intraluminal capsules are described and can be adapted for use in the instant embodiment. In an embodiment, circuitry and location sensors in the EADCD apply variable magnetic fields to steer the LTD within the airway by movement of the EADCD. In an embodiment to inspect a branch of the bronchial tree, the EADCD is moved laterally at a bronchial junction to steer the LTD down the lumen of the branch.

Steering of the LTD is guided by real time display of intra-bronchial images by the EADCD. Repeat imaging of the bronchial tree to reexamine tumors following chemotherapy may be guided by the landmark images stored in memory in the EADCD. To steer the LTD to the location of a tumor imaged previously, the landmark images (vasculature patterns from NIR sensing) at the tumor site may be recalled from EADCD memory. In an embodiment the stored intraluminal images previously transmitted by the LTD may be searched for images of the tumor and the corresponding linked landmarks (NIR patterns of subsurface vasculature) obtained by the EADCD are used to identify the tumor location.

The EADCD is moved over the body surface until the landmark pattern is located and the EADCD alerts the physician, caregiver, patient or other operator. The LTD is guided to the tumor site as the EADCD is moved over the body surface and displays the tumor site in real time once the location is reached. Comparison of images obtained before and after chemotherapy may indicate the status of the tumor, i.e., stable, shrinking, or growing. Removal of the LTD from the airway is accomplished by applying a variable magnetic field and moving the EADCD up the bronchial tube to the trachea. The LTD may be expelled by cough or retrieved with a bronchoscope, if the device is not biodegradable.

The lumen imaging system may be used to image putative tumor nodules ranging in size from 9-20 mm diameter (based on computed tomography (CT) scans) in order to evaluate the nodules. The patient is given a LTD to inhale, which is programmed to transmit data to an EADCD only upon arrival at a nodule site. Location sensors on the LTD signal arrival at a nodule site and alert the operator to move the EADCD over the LTD to allow image data transmission. Then with the EADCD the LTD is moved proximal to the next nodule on the CT scan and instructed to locate the nodule site using a location identifier (e.g., image analysis). The operator is alerted and moves the EADCD until it aligns with the LTD and image data is transmitted to the EADCD. In turn, each nodule site is found by the LTD using location identifiers and the corresponding landmark vascular pattern is imaged by the EADCD. Limited transmissions by the LTD conserve its battery and stored location identifiers (e.g., intraluminal images) and subsurface landmarks (e.g., vasculature patterns) allow returns to each of the nodules. Moreover, the stored nodule images are analyzed to determine malignancy, growth status and spread of putative tumor nodules.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. In an embodiment, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware. In some implementations described herein, logic and similar implementations can include software or other control structures. Electronic circuitry, for example, may have one or more paths of electrical current constructed and arranged to implement various functions as described herein. In some implementations, one or more media can be configured to bear a device-detectable implementation when such media hold or transmit device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, the various embodiments described herein can be implemented, individually and/or collectively, by various types of electro-mechanical systems having a wide range of electrical components such as hardware, software, firmware, and/or virtually any combination thereof and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Examples of electro-mechanical systems include but are not limited to a variety of consumer electronics systems, medical devices, as well as other systems such as motorized transport systems, factory automation systems, security systems, and/or communication/computing systems. Electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedia components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

This disclosure has been made with reference to various example embodiments. However, those skilled in the art will recognize that changes and modifications may be made to the embodiments without departing from the scope of the present disclosure. For example, various operational steps, as well as components for carrying out operational steps, may be implemented in alternate ways depending upon the particular application or in consideration of any number of cost functions associated with the operation of the system; e.g., one or more of the steps may be deleted, modified, or combined with other steps.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but may include other elements not expressly listed or inherent to such process, method, system, article, or apparatus.

In embodiments, the system is integrated in such a manner that the system operates as a unique system configured specifically for function of one or more of the systems described herein (e.g., with a described lumen traveling device, etc.), and any associated computing devices of the system operate as specific use computers for purposes of the claimed system, and not general use computers. In embodiments, at least one associated computing device of the system operates as a specific use computer for purposes of the claimed system, and not a general use computer. In embodiments, at least one of the associated computing devices of the system is hardwired with a specific ROM to instruct the at least one computing device. In embodiments, one of skill in the art recognizes that the systems described herein (e.g., with a described lumen traveling device, etc.) and associated systems/devices effect an improvement at least in the technological field of lumen traveling devices. Various embodiments described herein contribute to the medical field, specifically with diagnosis and/or treatment of disease, or maintenance of a healthy state by allowing visualization of internal locations within a subject that are not otherwise as easily accessible. In this regard, in an embodiment, a unique computer and/or system is required.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system, comprising:
a lumen traveling device communicably coupled with
an externally alignable display and control device;
wherein the lumen traveling device includes at least one means for locomotion, at least one power source, at least one of a transmitter, receiver, or transceiver, at least one location sensor;
wherein the externally alignable display and control device includes at least one of a transmitter, receiver, or transceiver; and
wherein a processor is operably coupled to at least one of the lumen traveling device or the externally alignable display and control device and is configured to receive signals from at least one of the lumen traveling device or externally alignable display and control device only upon the lumen traveling device entering at least one predetermined location threshold.

2. The system of claim 1, wherein the predetermined location threshold corresponds to a specific temporal or spatial threshold.

3. The system of claim 1, further including at least one transmitter to transmit at least one signal demarcating that the lumen traveling device is traveling in the at least one predetermined location threshold.

4. The system of claim 1, further including at least one memory device operably coupled to the processor.

5. The system of claim 1, further including means to align the externally alignable display and control device with the path previously traveled by the lumen traveling device, including at least one of at least one inertial sensor, at least one fiducial sensor, at least one topographical sensor, or at least one laser pointer.

6. The system of claim 5, wherein the sensor includes at least one of an imaging sensor, chemical sensor, pH sensor, time sensor, or accelerometer.

7. The system of claim 1, wherein the power source includes at least one battery.

8. The system of claim 7, wherein the battery includes at least one of a microbattery, nuclear battery, or thin film battery.

9. The system of claim 1, wherein the power source includes at least one of a fuel cell or biofuel cell.

10. The system of claim 1, wherein the power source includes at least one of a nanogenerator, optical power source, acoustic receiver, electromagnetic receiver, or electrical power source.

11. The system of claim 1, wherein the power source includes at least one of energy harvesting harvested internally, or wireless energy transfer.

12. The system of claim 11, wherein the energy harvesting is thermal energy harvesting, kinetic energy harvesting, or chemical energy harvesting.

13. The system of claim 12, wherein the kinetic energy harvesting includes harvesting energy from movement through a lumen, as a subject's body assists in locomotion of the lumen traveling device.

14. The system of claim 13, wherein the wireless energy transfer includes at least one of inductive energy transfer, capacitive energy transfer, or ultrasonic energy transfer.

15. The system of claim 1, wherein the means for locomotion includes a controller with control circuitry.

16. The system of claim 15, wherein the controller for the means for locomotion is programmable.

17. The system of claim 1, wherein the lumen traveling device is configured to be at least one of implanted, injected, ingested, or inhaled.

18. The system of claim 1, wherein the sensor includes one or more of an imaging sensor, a chemical sensor, a pH sensor, a time sensor, or an accelerometer.

19. The system of claim 1, wherein the lumen traveling device is a wireless endoscope capsule.

20. The system of claim 1, wherein the lumen traveling device is configured for use in a fluoroscopic procedure.

21. The system of claim 1, wherein the lumen traveling device is sized and shaped for traveling through at least part of one or more of a gastro-intestinal tract, blood vessel, urinary tract, genital tract, bronchial tube, nasal or sinus passage, ear canal, umbilical cord, or artificial lumen.

22. The system of claim 21, wherein the artificial lumen includes at least one of a catheter, or port.

23. The system of claim 22, wherein the first lumen traveling device includes at least one compartment containing at least one therapeutic agent or tag.

24. The system of claim 23, wherein the at least one tag includes at least one of a cellular stain, a nucleic acid stain, a protein stain, or a carbohydrate stain.

25. The system of claim 1, further including at least one memory device configured to store data associated with operation of the lumen traveling device.

26. The system of claim 1, wherein the processor is configured to determine at least one of velocity, speed, direction, or angle of travel for the lumen traveling device.

27. The system of claim 1, wherein the externally alignable display and control device includes at least one projector or display.

28. The system of claim 27, wherein the display includes at least one of an LED, LCD, or OLED display.

29. The system of claim 27, wherein the externally alignable display and control device is configured to be worn on at least a portion of a hand.

30. The system of claim 1, wherein the means for locomotion include at least one of a propulsion system, a hydrodynamic propulsion system, a fluid displacement system, a propeller, a paddle, a vibration system, a lumen wall-engaging system, or pneumatic bellow system.

31. The system of claim 1, wherein the means for locomotion include at least one of an actuator, a motor, a shape memory material, an electroactive material, a magnetic driver, an electronic driver.

32. The system of claim 1, wherein the means for locomotion include at least one steering means.

33. The system of claim 1, further including at least one controller configured to direct the lumen traveling device in response to the processor's determination based on the one or more sensed signals.

34. A method, comprising:
detecting at least one location signal of a lumen traveling device deployed in a lumen of a subject by way of one or more sensors on or in an externally alignable display and control device;
generating at least one response signal based on the detection of the at least one location signal;
determining if the at least one location signal exceeds a threshold value;
generating at least one communication signal based on the determination of whether the at least one location signal exceeds a threshold value.

35. The method of claim 34, wherein determining if the at least one location signal exceeds a threshold value includes comparing the at least one location signal to a reference data indicative of the threshold value.

36. The method of claim 35, wherein the reference data includes at least one second location signal detected.

37. The method of claim 35, wherein determining if the at least one location signal exceeds a threshold value includes determining the location of the lumen traveling device.

38. The method of claim 35, wherein determining if the at least one location signal exceeds a threshold value includes evaluating whether the location of the lumen traveling device has entered a predetermined target location.

39. The method of claim 34, wherein the lumen traveling device includes electronic circuitry operably coupled to the one or more sensors and an externally alignable display and control device, the electronic circuitry configured to instruct the lumen traveling device to alter at least one parameter in response to receiving at least one sensed signal from the one or more sensors.

40. The method of claim 39, wherein the at least one parameter includes changing one or more of direction of travel, speed of travel, image capture of the lumen, release of at least one therapeutic agent or tag, or changing at least one mode of output.

41. The method of claim 35, further including storing in memory, sensed signals from the one or more sensors of the lumen traveling device.

42. The method of claim 35, wherein the detecting at least one location signal of a lumen traveling device deployed in a lumen of a subject by way of one or more sensors on or in the lumen traveling device is by way of an externally alignable display and control device.
